(19) United States
(12) Patent Application Publication
(10) Pub. No.: US 2009/0036527 A1
(43) Pub. Date: Feb. 5, 2009

(54) Therapeutic Application Of Leonurine In Treating Cardiomyopathy

(76) Inventors: YIZHUN ZHU, Singapore (SG); Xinhua Liu, Shanghai (CN); Yichun Zhu, Shanghai (CN); Aijun Hou, Shanghai (CN); Xun Sun, Shanghai (CN)

Correspondence Address: YUAN QING JIANG, P.O. BOX 61214, PALO ALTO, CA, 94306, US

(21) Appl. No.: 12/185,052

(22) Filed: Aug. 1, 2008

(30) Foreign Application Priority Data
Aug. 2, 2007 (CN) CN 200710044524.0

(51) Int. Cl. A61K 31/221; C12N 5/06; A61P 9/10

(52) U.S. Cl. 514/534; 435/375

(57) ABSTRACT

The present invention provides methods for treating ischemic cardiomyopathy. In one embodiment, provided is a method of treating a disease characterized as ischemic cardiomyopathy, comprising administering to a subject suffering from said disease a pharmaceutical composition comprising therapeutically effective amount of synthesized Leonurine. The method induces at least one biochemical change to improve hypoxia myocardial cells survive rate, reduce LDH releasing from the ischemic myocardial cells, and reduce infarction area of cardiomyopathy in the subject.

### FIELD OF INVENTION

The present invention relates to methods of therapeutic applications of Leonurine in treating ischemic cardiomyopathy.

### BACKGROUND OF INVENTION

Ischemic cardiomyopathy, especially myocardial infarction is irreversible necrosis of myocardial cells caused by ischemic heart disease, which is characterized by coronary blood flow diminished to the level unsustainable to the metabolic demand of myocardium.

Common symptoms of ischemic heart disease include angina, shortness of breath, or fatigue. Often angina is worsened if the patient exerts after a meal, or walks into a cold weather, or suffers from emotional stress.

The major pathogenesis of ischemic myocardial infarction is coronary artery stenosis, leading to myocardial cells starved for oxygen (hypoxia) and glucose, the result of which is death or permanent damage of myocardial cells (myocytes).

The etiologies for coronary artery stenosis are fixed atherosclerotic obstruction, acute plaque rupture, coronary artery thrombosis, and vasospasm. In order to study ischemic myocardial infarction, lab models are established by occlusive ligature of coronary artery of experimental animals to reproduce ischemia caused by coronary artery stenosis, or by deprivation oxygen and glucose supply to cultured myocardial cells.

Morphologically the area of necrotic myocardium corresponds to the area where occluded coronary artery supplies. The myocardial tissue affected turns from pallor to cyanotic, further to softened yellow central area with a hyperemic rim. Eventually the dead myocardial tissue is replaced by a fibrotic white thin scar. The severity of the damage to myocardium is proportional to the area affected by coronary artery stenosis and the time duration of ischemia.

Under microscope the ischemic myocardial cells display various morphological changes ranging from myocytolysis, eosinophilic cell infiltration with intercellular edema of the myocardium, acute inflammation of the myocytes, macrophages removing dead myocytes, granulation tissue, to scar tissue.

Biochemical lab diagnosis provides specific, sensitive and timely results indicating myocardial cell stress, injury, and death. The lab test markers relevant to myocardial cell's current biomedical conditions are creatine kinase (CK) level, creatine kinase sub-fraction MB (CK-MB) level, cardiac troponin levels (troponin-T and troponin-I), Lactate Dehydrogenase (LDH) level and myoglobin level.

Elevation of CK or CK-MB indicates acute myocardial cell injury, since it is a specific enzyme in myocardial cells and a good marker of injury of myocardial cells. Isoforms 1 and 2 of CK-MB can also be tested, and the ratio of the two CK-MB isoforms can provide further information about the injury condition of myocardial cell.

Troponin-T and Troponin-I are proteins in myocardial cells. Elevation of the Troponin-T and Troponin-I indicate that myocardial cells are injured.

Elevation of LDH level is another indicator of myocardial infarction.

Myoglobin is structure protein of myocytes. Increase of its level indicates myocardial infarction.

Numerous drug treatments to combat coronary heart disease have been developed. Commonly prescribed drugs to treat coronary diseases are beta-blockers, nitrates, calcium channel blockers, angiotensin-converting enzyme (ACE) inhibitors, and antiplatelet coaggregation drugs.

Beta-blockers are prescribed to alleviate the effect of adrenaline and noradrenaline on the heart. Nitroglycerin dilates coronary blood vessels instantly. Calcium channel blockers prevent blood vessels from constricting and counter coronary artery spasm. ACE inhibitors, such as ramipril reduce the risk of heart attack. Antiplatelet coaggregation drugs, such as aspirin reduce the aggregation of platelets so that they do not clump and stick on blood vessel walls. Some of the drugs are used in combination to prevent or reduce ischemia and to minimize symptoms.

Searching for new drugs to treat coronary heart disease has been an on going effort worldwide. Natural resources have been the dependable sources for new drug development for long time. New drugs developed from substances originated from plants are believed less dependent forming, with fewer side effects.

### SUMMARY OF THE INVENTION

The present invention provides therapeutic applications of Leonurine in treating ischemic cardiomyopathy.

In particular, and by way of example only, according to an embodiment, provided is a method of treating a disease characterized as ischemic cardiomyopathy, comprising administering to a subject suffering from said disease a pharmaceutical composition comprising therapeutically effective amount of synthesized Leonurine of Formula I:

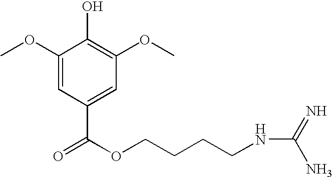

It is to be understood that both the foregoing general description and the following detailed description are exemplary and explanatory and are intended to provide further explanation of the invention as claimed.

### BRIEF DESCRIPTION OF THE DRAWINGS

FIG. 1 shows effects of Leonurine on survival rate of myocardial cells (cell viability percentage) under hypoxia condition.

FIG. 2 shows effects of Leonurine on LDH leakage (release) of myocardial cells under hypoxia condition.

FIG. 3 shows effects of Leonurine on Catalase activity of myocardial cells under hypoxia condition.

FIG. 4 shows effects of Leonurine on SOD and CuZn-SOD activities of myocardial cells under hypoxia condition.

FIG. 5 shows effects of Leonurine on MDA content of myocardial cells under hypoxia condition.

FIG. 6 shows effects of Leonurine on infarct size (%) of myocardial ischemic rat and mortality rate (%) of rat.

FIG. 7 shows effects of Leonurine on LDH level in plasma of myocardial ischemic rat.

FIG. 8 shows effects of Leonurine on CK level in plasma of myocardial ischemic rat.

FIG. 9 shows effects of Leonurine on MDA level in plasma of myocardial ischemic rat.

FIG. 10 shows effects of Leonurine on SOD level in left ventricle of myocardial ischemic rat.

FIG. 11 shows effects of Leonurine on mRNA level of Bcl-2 and Bax (fold differences) of myocardial ischemic rat.

FIG. 12 shows effects of Leonurine on Bcl-2 and Bax protein levels (densities) of myocardial ischemic rat.

### DETAILED DESCRIPTION OF THE INVENTION

For long time, Chinese Motherwort (Herba Leonuri) has been used in China for treating woman's conditions. Chinese believe that taking Motherwort helps relieving blood stasis. Motherwort has been given to women who have irregular menstruation such as amenorrhea or dysmenorrhea, and to women who are giving birth to children to relax the uterus and help the labor. After childbirth, Motherwort is given to women again to help the recovery of the uterus. Besides for treating women's disease, Chinese have been using Motherwort as a diuretic to treat edema, such as edema caused by acute renal inflammation.

Ancient Greeks and Romans believed that a similar species of Motherwort (Leonurus cardiaca), which is often called by its common names Lion's Tail, Lion's Ear, Throw Wort, Roman Motherwort or Leonurus, has sedative effect and can be used as an antidepressant or pain reliever. Some herb practitioners also use Motherwort on various disorders ranging from heart diseases to liver diseases.

Because of its long time and ubiquitous use as a cure-all herb medicine, and because of its low toxic side effects apparently, researchers are interested in Motherwort's chemical compositions and their mechanisms of treating diseases in central nervous system, cardiovascular system and woman's reproductive system.

For example, Dr. Zhu Yizhun has discovered that, Kardigen, a purified compound from Mortherwort, has anti-oxidative effects and can prevent ischemia cardiomyopathy effectively (Acta Physiologica Sinica, Oct. 25, 2007, 59 (5): 578-584).

Patent application WO2008031322 and Chinese patent application 200610107041.6 disclose leonurus extractive can be applied as acetylcholine esterase inhibitor because of its cholinomimetic effect, and can be used to treat many diseases. Among the methods of treating the diseases, the applicants disclose a method to inject Chinese Motherwort extract containing active ingredients to protect myocardium from ischemic myopathy. In the method the applicants state that Chinese Motherwort extract, which containing at least five ingredients, can be used to reduce MDA content, increase activities of SOD and GSH-PX (Glutathione peroxidase) in myocardial tissue, and improve ischemic situation demonstrated on EKG. The protection of myocardium, according to the applicants of WO2008031322, is through the effect of Chinese Motherwort of protecting anti free radical enzyme system activity, inhibiting lipid reaction at reperfusion of ischemic myocardium, and reducing damage from excessive oxygen free radicals. However, the ingredient which is responsible to protect ischemic myocardium from damage is unknown and not understood.

More compositions in Motherwort are isolated and purified. Leonurine, which is a plant alkaloid purified from Chinese Motherwort, is drawing attention from researches and chemists.

For example, it is reported that Leonurine inhibits intracellular calcium ion release and outside calcium ion influx to vascular smooth muscle cells through calcium channels, thereby inhibiting the contraction of the smooth muscle cells. (Chang-Xun Chen, Chiu-Yin Kwan. Endothelium-independent vasorelaxation by leonurine, a plant alkaloid purified from Chinese motherwort. Life Sciences 68 (2001) 953-960)

Zhao Wang et al. disclose the effects of Leonurine on the activity of creatine kinase (CK). Leonurine inhibits the enzyme activity in concentration- and time-dependent manners (Journal of Asian Natural Products Research, Volume 6, Number 4, December, 2004, pp. 281-287(7)).

Chemists have determined the chemical structure of Leonurine and the compound has been synthesized successfully.

Chinese patent application 02138220 published on May 7, 2003 discloses a method of synthesizing a salt of Leonurine.

Cheng KF et al. disclosed an improved synthesizing method for Leonurine. And now synthesized Leonurine can be purchased from market place, such as from General Enterprise Corporation of University of Science and Technology, or from Anhui New Star Pharmaceutical Development Co., Ltd (http://www.newstar-chem.com/english/display.asp!id=208).

Despite many research disclosures, there are no reports about the application of Leunurine on treating ischemic cardiomyopathy so far.

In one embodiment according to the present invention, provided is a novel method to apply Leonurine to treat ischemic cardiomyopathy. Particularly, a pharmaceutical composition comprising therapeutically effective amount of Leonurine and at least one pharmaceutically acceptable carrier is prepared for treating a subject suffering from a disease characterized as ischemic cardiomyopathy.

The chemical structure of Leonurine is shown as the following (Formular I): C₁₄H₂₁N₃O₅

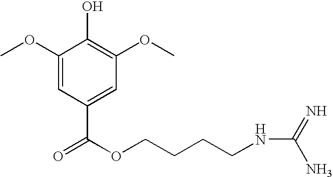

The structure of Leonurine is capable of forming pharmaceutically acceptable salts, including acid addition salts and base salts, as well as solvates, such as hydrates and alcoholates. All of these pharmaceutical forms are contemplated by this invention and are included herein.

Pharmaceutically acceptable acid addition salts of the composition of Leonurine include salts derived from inorganic acids such as hydrochloric, nitric, phosphoric, sulfuric, hydrobromic, hydriodic, and the like, as well as the salts derived from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts include nitrate, phosphate, monohydrogenphosphate, dihydrogenphosphate, metaphosphate, pyrophosphate, chloride, bromide, iodide, acetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like.

The phrase “pharmaceutically acceptable” is employed herein to refer to compositions and dosage forms suitable for use in contact with the tissues of human beings and animals without excessive toxicity, irritation, allergic response, or other problem or complication, commensurate with a reasonable benefit versus risk ratio.

The compound Leonurine of the invention may be formulated into pharmaceutical compositions by admixture with pharmaceutically acceptable non-toxic excipients or carriers. Common excipients or carriers may be sterile water or saline, polyalkylene glycols such as polyethylene glycol, oils from vegetable origin, hydrogenated naphtalenes etc. Such compounds or compositions may be prepared for parenteral administration intraveneously, subcutaneously, and intramuscularly, particularly in the form of liquid solutions or suspensions in aqueous physiological buffer solutions; for oral administration, particularly in the form of tablets or capsules; or for intranasal administration, particularly in the form of powders, nasal drops, or aerosols. Sustained release compositions are also encompassed by the present invention. Other suitable administering systems include ethylene-vinyl acetate copolymer particles, osmotic pumps, implantable infusion systems, and liposomes. Compositions for other routes of administration may be prepared as desired using standard methods.

In an alternate embodiment, the invention relates to compositions and kits comprising a first therapeutic agent including Leonurine thereof and at least one of second therapeutic agent. The second therapeutic agent is not Leonurine or its analogues thereof. These compositions or kits are effective to treat ischemic cardiomyopathy in a subject. Various therapeutic agents, including beta-blockers, nitrates, calcium channel blockers, angiotensin-converting enzyme (ACE) inhibitors, and antiplatelet coaggregation drugs may be used in the composition.

The effectiveness of Leonurine on treating myocardial infarction is disclosed in both cultured myocardial cells and animal models.

Myocardial cells are deprived from oxygen (hypoxia) and glucose for 6 hours, which is a conventional model for ischemic cardiomyopathy. The ischemic myocardial cells are then reperfused with normal amount of oxygen and glucose (hypoxia/reoxygenation process). Several groups of ischemic cells are administered with Leonurine respectively before and during hypoxia/reoxygenation process, with one group of ischemic cells left untreated (No Leonurine is administered.) as a positive control of myocardial infarction (vehicle (MI) group) and one control group of myocardial cells cultured without hypoxia/reoxygenation process.

Losartan is an angiotensin receptor blocker. Angiotensin formed in the blood by the action of angiotensin converting enzyme (ACE). Angiotensin causes vasoconstrict by binding to angiotensin receptors on smooth muscle cells of blood vessels, leading to hypertension. Losartan blocks the angiotensin receptor thereby reduces blood pressure. In the experiments of this invention disclosure, Losartan is a positive control drug used for comparison with the effect of Leonurine.

The effects of Leonurine inducing biochemical changes in the ischemic or hypoxic myocardial cells, the survival rate of the ischemic or hypoxic myocardial cells and the LDH leaking from the ischemic or hypoxic myocardial cells are observed. The results indicate LDH leaking from the ischemic or hypoxic myocardial cells is reduced by Leonurine treatment.

Several biochemical markers are assessed to determine the effects of Leonurine. In ischemic or hypoxic myocardial cells treated with Leonurine, LDH leakage (release) from the cells is reduced. The result indicates that the reduction of LDH leaking rates in Leonurine treated cells are correlated with increase of the survival rate of myocardial cell under hypoxia condition.

The activities of SOD (superoxide dismutase) in Leonurine treated ischemic or hypoxic cells are elevated. The result indicates that the elevating SOD activity in Leonurine treated cells are correlated with increase of the survival rate of myocardial cell under hypoxia condition. SOD subtype studies further reveal that Leonurine are able to elevate the activities of Cu—Zn SOD in cytoplasm in myocardial cell under hypoxia condition.

Catalase activity in Leonurine treated ischemic or hypoxic cells groups is elevated.

Lipid peroxidations in ischemic or hypoxic cells treated with Leonurine are inhibited by it. Leonurine can significantly inhibit oxygen free radicals in the cells, thereby inhibiting lipid peroxidation in ischemic or hypoxic myocardial cells, reducing the level of MDA (malondialdehyde), which is a product of lipid peroxidation.

Leonurine can inhibit myocardial cell apoptosis, and have great value in application of treating heart disease.

Animal model for ischemic myocardiopathy is employed and effectiveness of Leonurine in treating ischemic myocardial infarction (MI) is tested.

Rats are randomly assigned to 4 experiment groups: Sham operated on and treated with saline group (Control), ligation operated on and treated with saline (vehicle) group (MI group), ligation operated on and treated with Leonurine groups (7.5 mg/kg/day), ligation operated on and treated with Leonurine groups (15 mg/kg/day). Rats in all treated groups are pre treated with Leonurine respectively for 7 days.

Rats are operated on and MI is induced according to correspondent experiment groups by ligating the left anterior descending coronary artery at approximately 2-3 mm from its origin. The MI model is established when the area of myocardium supplied by the ligated coronary artery turns to pallor, and ECG recording shows the ST segment is elevated. Then the rats are given water and food, returned to their cages according to experiment groups. All treatment groups are given Leonurine continuously for 2 more days.

Forty eight hours after the surgery, ECG is recorded for each rat in each group. Blood samples are taken from abdominal aorta from each rat in each group. Then the rats are sacrificed and their hearts are taken. The heart tissues from the rats are stained and the myocardial infarction areas are observed.

To evaluate the effect of Leonurine on MI, infarct size of heart tissue are measured after TTC staining (FIG. 12). The infarct size of left ventricular area was significantly less in rats subjected to Leonurine treatments than in vehicle injected rats. However, differences in ECG patterns were similar in all groups prior to the start of the treatment as well as one week after treatment (FIG. 13).

Serum creatine kinase (CK) levels and LDH leakages are decreased after Leonurine treatment of the animals. Lipid peroxidation reduced since Leonurine inhibit creation of oxygen free radicals therefore inhibit lipid peroxidation, so that the product of lipid peroxidation MDA content decreased. SOD (superoxide dismutase) activity is elevated in Leonurine treated animals.

Bax expression, which is related to cell injuries and apoptosis, is down regulated in animal groups treated with Leonurine. Bcl-2 expression, which is anti-apoptotic, is up regulated.

The expressions of Bcl-2 and Bax mRNAs confirm that the effects of Leonurine on the animals.

Heart failure (HF) animal model is employed. HF model is occlusive ligation of anterior descending coronary artery of a rat, after 8 weeks, HF develops and the rat HF model is established. Leourine is given via intragastric administration to the rat.

Detailed procedure is described as the following: HF is induced by ligation of the left anterior descending coronary artery at approximately 2-3 mm from its origin. ECG is recorded in the anaesthetized animal for a period of one minute as controls. The proximal left anterior descending coronary artery, which supplies blood to left ventricle of the heart, is ligated at the position 2-3 mm from the aorta with a 5-0 atraumatic suture that is passed through the superficial layers of myocardium, between the left auricle and the cone of pulmonary artery. The HF model is considered completely established when the area of myocardium supplied by the ligated coronary artery turns to pallor, and ECG recording show the ST segment is elevated. Then incisions are sutured and the chests are closed. Sham operated rats are prepared in the same manner except the left coronary is not ligated. After completion of the surgical procedures, rats are removed from the ventilator. The rats are kept warm, given water and food after they are awake from the anesthesia.

Two days after ligation, surviving rats are performed in four groups of rats in random fashion. Rats are randomly assigned to four treatment groups: group (1) Sham-treated with saline (Sham, control), (2) HF treated with saline, (3) HF treated with Leonurine (15 mg/Kg/day), (4) HF treated with Leonurine (30 mg/Kg/day). All treatment was given via intragastric administration. The experimental period was 8 weeks.

Eight weeks after the surgery, ECG is recorded for each rat in each group. Blood samples are taken from abdominal aorta from each rat in each group.

To evaluate the effect of Leonurine on HF, catheterizations to arteries and ventricles are performed to record cardiac function such as heat rate (HR), Mean Aortic Pressure (MAP), Left Ventricular Systolic Pressure (LVSP), Left Ventricular End-Diastolic Pressure (LVEDP), peak positive first derivative of left ventricular pressure (+dP/dt), peak negative first derivative of left ventricular pressure (−dP/dt). Data from the measurement of the recording were analyzed to determine the effect of Leonurine. Furthermore, plasma cysteine level and plasma ascorbic acid level were measured by capillary electrophoresis.

The results shown in Table 1 indicate that Leonurine can reduce LVEDP, increase speed of contraction and improve cardiac function, thereby Leonurine is effective on treating ischemic cardiomyopathy or ischemic MI.

Plasma measurements of Leonurine level, cysteine level and ascorbic acid (Vitamin C) shown in Table 2 indicate that Leonurine can reduce plasma cysteine level and increase plasma ascorbic acid level.

Plasma cysteine level and ascorbic acid level are biochemical markers indicating ischemic cardiomyopathy or ischemic MI condition. It is conceivable that Leonurine can be applied for increasing the plasma ascorbic acid level and reducing cysteine level in a subject, such as a human patient, thereby Leonurine is effective on treating ischemic cardiomyopathy or ischemic MI.

Since the structure of Leonurine is capable of forming pharmaceutically acceptable salts, including acid addition salts and base salts, as well as solvates, such as hydrates and alcoholates, the application of Leonurine to the subject to increase ascorbic acid level or reduce cysteine level will be in a pharmaceutical composition comprising therapeutically effective amount of synthesized Leonurine and at least one pharmaceutically acceptable carrier. All of these pharmaceutical forms are contemplated by this invention and are included herein.

In summery, Leonurine is effective to treat ischemic cardiomyopathy or ischemic myocardial infarction by inducing a number of biochemical changes, which is manifested in biochemical marker changes.

### EXAMPLES

### Example 1A

### Leonurine's Effect on Improving Survival Rate of Myocardial Cells After Hypoxia

The heart sample of 3 day old Sprague-Dawley (SD) rat (first generation) was washed in PBS (phosphate balanced solution) under sterilized condition. The sample is digested in 0.08% pancreatic enzyme solution 37 degree C for 10 minutes in a flask, wherein the solution was stirred constantly. In order to stop the digestion, serum was added into the flask and mixed with the solution. The digesting process was repeated 8 times, and the supernatant of each digestion was collected. The supernatant was centrifuged 2,000/minute for 5 minutes, and the myocardial cells were collected each time. The myocardial cell density was adjusted to 10⁶/sample and cultured in DMEM containing 10% calf serum for 3 days.

The cultured myocardial cells were divided into the following groups:

Control group, no Leonurine treatment, no hypoxia process.

Hypoxia (vehicle, MI) group, no Leonurine treatment, under hypoxia condition for 6 hours.

Leonurine group, under hypoxia condition for 6 hours then cultured with corresponding treatment of Leonurine 10⁻⁶ mol/L.

Losartan (positive control) group, under hypoxia condition for 6 hours t with corresponding treatment of Losartan 10⁻⁶ mol/L.

Myocardial cell viability was assessed by MTT method, which is the measurement of the reduction of 3-(4,5-dimethylthiazol-2-yl)-2,5-diphenyltetrazolium bromide (MTT). MTT (0.5 mg/ml) was added to 100 μl of each well (8×10³ cells/well in 96-well plates), cultured for 4 hr and a dark blue formazan (dye) product was produced in the culture. The amount of formazan was measured using a microplate reader at spectrum 570 nm.

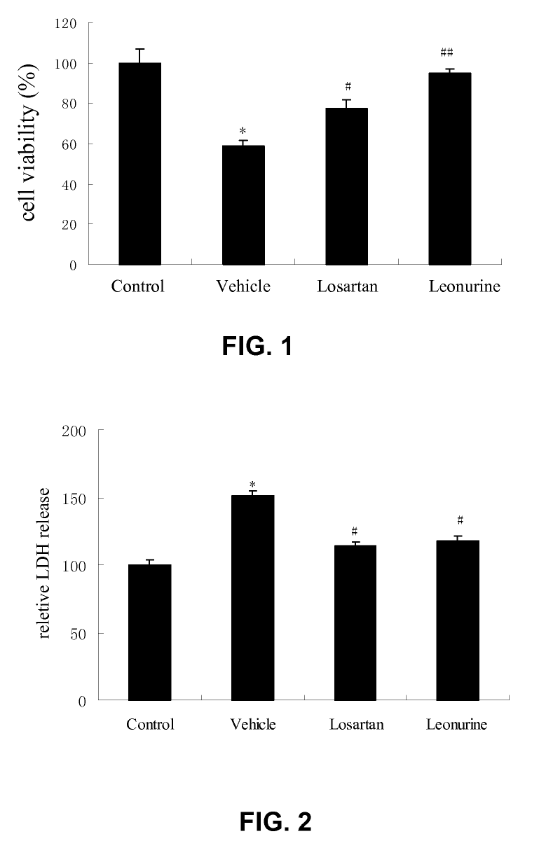

The result (FIG. 1) shows that myocardial cell survival rate in MI (vehicle) group was lower than that of control group. Leonurine can significantly increase the survival rate of myocardial cell under hypoxia condition. The difference was p<0.01 statistically significant by single factor Chi square analysis (Pearson's chi-square test). (The symbol * indicates the difference is significant (p<0.05), # indicates the difference between Leonurine and Losartan experiments is significant(p<0.05) and ## indicates the difference between Leonurine and Losartan experiments is significant (p<0.01).)

### Example 1B

### Leonurine's Effect on Lowering LDH Leakage from Hypoxia Injured Myocardial Cells

Cell death leads LDH leakage (release) from the cell. Leonurine can reduce LDH leakage from inured myocardial cells thereby increase the survival rate of the myocardial cells.

Myocardial cell samples were obtained from the same procedure of Example 1A, the experiment groups were divided and hypoxia experiments were performed on Control group (no Leonurine treatment, no hypoxia/reoxygenation process), vehicle (hypoxia) group (no Leonurine treatment, under hypoxia condition for 6 hours), and Leonurine group (10⁻⁶ mol/L). Myocardial cells were obtained from each sample (10⁶ myocardial cells/sample) from the Control group, vehicle (hypoxia) group, Leonurine group. The cells were lysed under sterilized condition, and LDH (Lactate Dehydrogenase) contents in each sample of the cells were tested by pyruvic acid production method, using LDH content in Control sample cells as standard of 100%. The rate of LDH leakage (release) was calculated, which is the difference between LDH content of Control group samples and vehicle (hypoxia) group or Leonurine group or Losartan group.

The result (FIG. 2) showed that LDH leaking (release) rates in Leonurine group and Losartan group groups were lower than that of vehicle (hypoxia) group, the differences were p<0.01 statistically significant by single factor Chi square analysis. Therefore Leonurine can significantly lower LDH leaking rates from myocardial cell under hypoxia process.

### Example 1C

### Leonurine's Effect on Elevating the Activities of Catalase and Inhibiting the Creation of Free Radicals in Hypoxia Injured Myocardial Cells

Several other biochemical markers are assessed to determine the effects of Leonurine.

Myocardial cell samples were obtained from the same procedure of Example 1B, and the experiments were performed same as Example 1B with the same dosages and same conditions.

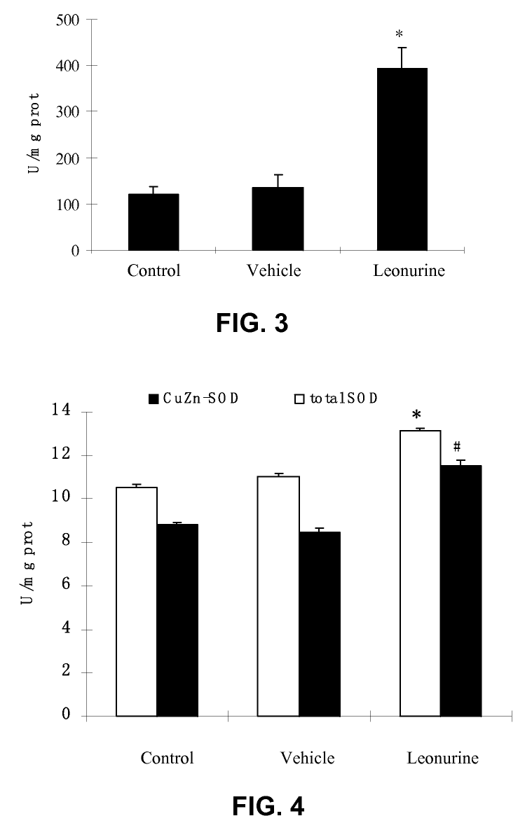

Catalase can catalyze oxygen free radicals, its activity in Leonurine treated hypoxia cells groups is elevated. (FIG. 3)

SOD (superoxide dismutase) plays an important role in balancing oxidant and antioxidant system in an organism. SOD can eliminate superoxide free radicals, thereby protect cells from injury. The activities of SOD and CuZn-SOD in Leonurine treated hypoxia cells are elevated. The result indicates that the elevating SOD and CuZn-SOD activities in Leonurine treated cells are correlated with the survival rate of myocardial cell under hypoxia condition. (FIG. 4)

Oxygen free radicals produced by enzyme and non enzyme systems in an organism attack poly unsaturated fatty acid in biomembrane, inducing lipid peroxidation and producing MDA (malondialdehyde). MDA can react with thiobarbituric acid (TBA), forms red product, which can be detected in spectrophotometer at 532 nm. The level of MDA can be used to indicate lipid peroxidation, thereby reflect condition of cell injury.

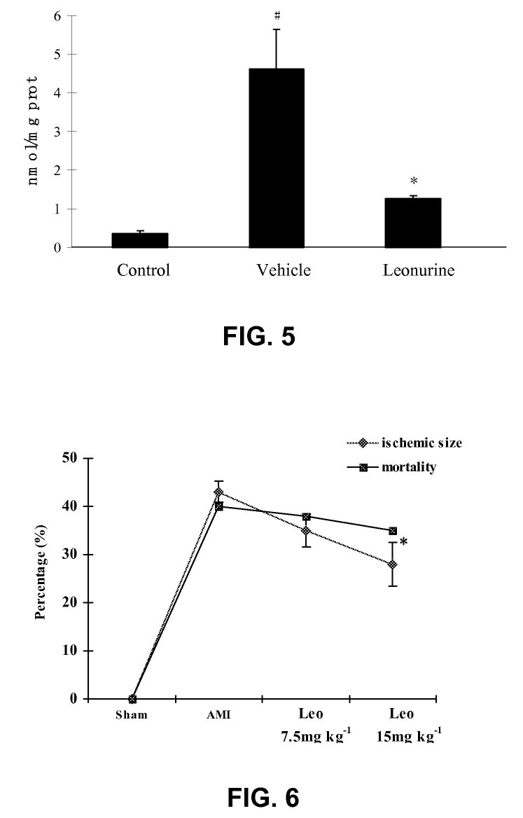

MDA level from lipid peroxidation in hypoxia cells treated with Leonurine was inhibited by Leonurine, indicating lipid peroxidation was reduced. (FIG. 5)

Leonurine can protect myocardial cell from ischemic injury, and have great value in application of treating ischemic myocardiopathy.

### Example 2A

### Effect of Leonurine on Infarct Size and Mortality in Rats Following MI Injury

MI animal model, which was occlusive ligation of anterior descending coronary artery, was employed and Leourine was intraperitoneal injection to observe the effect of Leonurine.

Leonurine was dissolved in saline (vehicle). Male SD rats (weight 200-250 g) were randomly divided to four treatment groups: group (1) Sham-operated with saline (Sham, control), (2) MI treated with saline (AMI), (3) MI treated with Leonurine (7.5 mg/Kg/day), (4) MI treated with Leonurine (15 mg/Kg/day). Rats were pre-treated for seven days with corresponding dosages of Leonurine respectively via an intraperitoneal injection once daily before they were used for MI model studies.

MI was induced on day eight by ligation of the left anterior descending coronary artery at approximately 2-3 mm from its origin. Briefly, the rats were anesthetized with 7% choral hydrate (60 mg/kg), endotracheally intubated and mechanically ventilated with room air, respiratory rate 100 breaths/min, tidal volume 2.5 ml with a rodent ventilator (DHX-150, China). ECG was recorded in the anaesthetized animal for a period of one minute using the Animal Mflab200 amplifier (Produced in China) as controls. A left thoracotomy was performed and the third intercostal space was exposed. The proximal left anterior descending coronary artery, which supplies blood to left ventricle of the heart, was ligated at the position 2-3 mm from the aorta with a 5-0 atraumatic suture that was passed through the superficial layers of myocardium, between the left auricle and the cone of pulmonary artery. The MI model was considered completely established when the area of myocardium supplied by the ligated coronary artery turned to pallor, and ECG recording showed the ST segment was elevated. Then incisions were sutured and the chests were closed. Sham operated rats were prepared in the same manner except the left coronary was not ligated. After completion of the surgical procedures, rats were removed from the ventilator and the endotracheal tube removed. The rats were kept warm, given water and food after they were awake from the anesthesia and kept in different cages according to experiment groups. The rats in correspondent experiment groups were given corresponding dosages of Leonurine continuously for 2 more days, while the rats in control and MI groups were given saline.

Forty eight hours after the surgery, ECG was recorded for each rat in each group. Blood samples were taken from abdominal aorta from each rat in each group. Then the rats were sacrificed and their hearts were taken, put into TTC solution, pH 7.4 at 37 degree C for 15 minutes. The heart tissues from the rats were stained and the myocardial infarction areas are observed.

To evaluate the effect of Leonurine on MI, infarct size were measured after TTC staining, and rat mortality rates were calculated (FIG. 6). The infarct size of myocardium was significantly less in rats subjected to Leonurine treatments than in saline (vehicle) injected rats, and the mortality rates in rats subjected to Leonurine treatments were not difference in saline (vehicle) injected rats.

### Example 2B

### Effect of Leonurine on Lactate Dehydrogenase (LDH) Leakages, Creatine Kinase (CK) Activity in Plasma.

MI injuries myocardial cells and CK and LDH levels are increased in the serum of the animals. After MI, the animal plasma CK level and LDH level were measured, and Leonurine's effects were assessed.

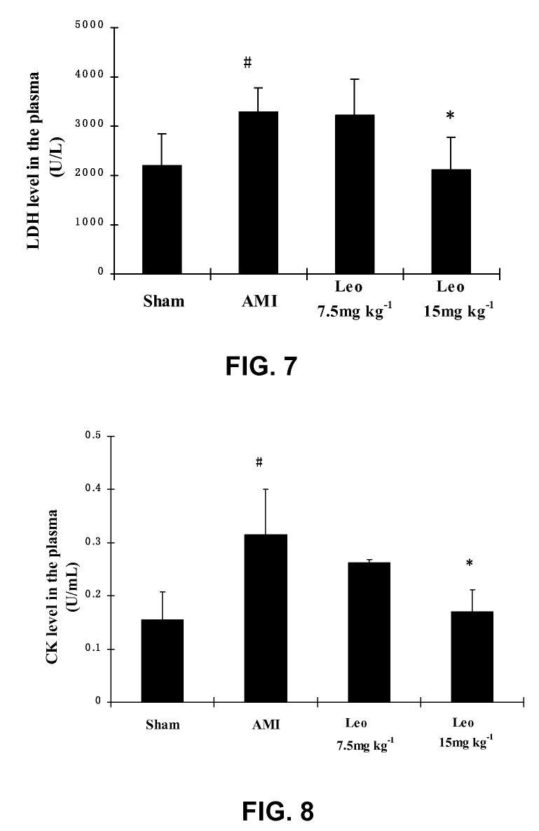

Same MI models in same rat groups as Experiment 2A were established. Plasma CK levels were detected with diagnostic kit (NJBI, China) according to the instructions. LDH were determined colorimetrically with a spectrophotometer. The result indicate that Leonurine can significantly reduce plasma LDH and CK levels, indicating that it can alleviate the severity of the MI injury to myocardial cells (p<0.05). (FIG. 7 and FIG. 8)

### Example 2C

### Effect of Leonurine on Malondialdehyde (MDA) Levels in Plasma and Superoxide Dismutase (SOD) Activity in Myocardium

MDA reflect lipid peroxidation level in MI injuries myocardial cells. SOD plays an important role in balancing oxidant and antioxidant system in an organism. SOD can eliminate superoxide free radicals, thereby protect cells from injury.

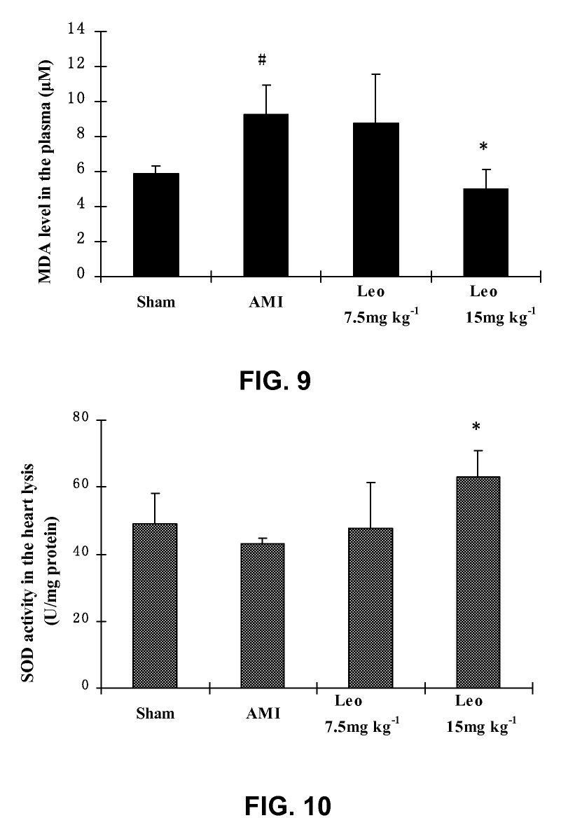

Same MI models in same rat groups as Experiment 2A were established. Plasma MDA level and left ventricle SOD activity were detected. The result indicate that Leonurine can significantly reduce plasma MDA level, and increase SOD activity. (FIG. 9 and FIG. 10)

### Example 2D

### Effect of Leonurine on the Expression of Bcl-2 and Bax mRNA and Protein Levels

Bax is a gene promoting cell apoptosis. Bcl-2 is an anti apoptosis gene.

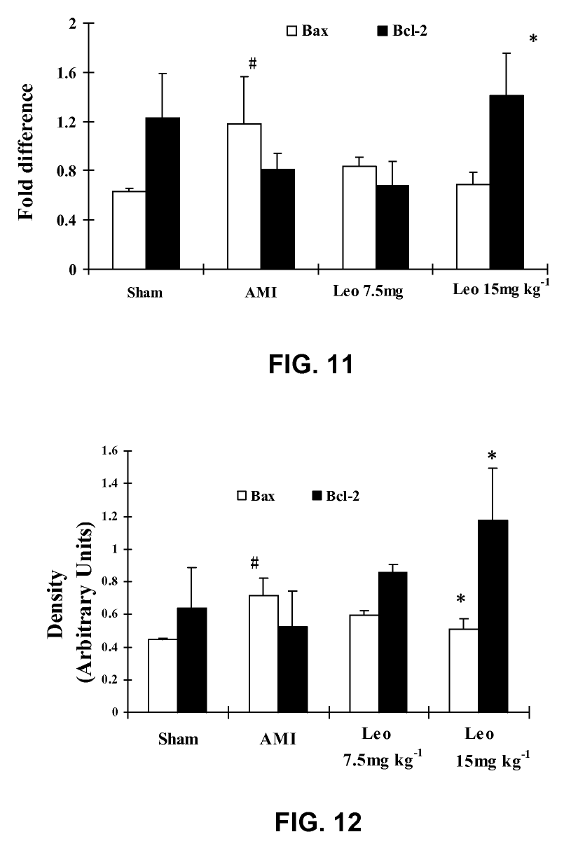

Same MI models in same rat groups as Experiment 2A were established. mRNA and protein levels of Bax and Bcl-2 were detected. The result indicate that Leonurine can significantly reduce Bax mRNA and protein expression, and increase Bcl-2 mRNA and protein expression. (FIG. 11 and FIG. 12)

The results of the experiments indicate that Leonurine is able to protect ischemic myocardial cells and can be used to prepare drugs to treat ischemic myocardiopathy.

### Example 3

### Effects of Leonurine on Cardiac Function, Plasma Cysteine Level and Ascorbic Acid Level in Animal Heart Failure Model

Heart failure (HF) animal model, which was occlusive ligation of anterior descending coronary artery, was employed. All treatment was given via intragastric administration. The experimental period was 8 weeks.

HF was induced on day eight by ligation of the left anterior descending coronary artery at approximately 2-3 mm from its origin. Briefly, the rats were anesthetized with 7% choral hydrate (60 mg/kg), endotracheally intubated and mechanically ventilated with room air, respiratory rate 100 breaths/min, tidal volume 2.5 ml with a rodent ventilator (DHX-150, China). ECG was recorded in the anaesthetized animal for a period of one minute using the Animal Mflab200 amplifier (Produced in China) as controls. A left thoracotomy was performed and the third intercostal space was exposed. The proximal left anterior descending coronary artery, which supplies blood to left ventricle of the heart, was ligated at the position 2-3 mm from the aorta with a 5-0 atraumatic suture that was passed through the superficial layers of myocardium, between the left auricle and the cone of pulmonary artery. The MI model was considered completely established when the area of myocardium supplied by the ligated coronary artery turned to pallor, and ECG recording showed the ST segment was elevated. Then incisions were sutured and the chests were closed. Sham operated rats were prepared in the same manner except the left coronary was not ligated. After completion of the surgical procedures, rats were removed from the ventilator and the endotracheal tube removed. The rats were kept warm, given water and food after they were awake from the anesthesia and kept in different cages according to experiment groups. The rats in correspondent experiment groups were given corresponding dosages of Leonurine continuously for 8 weeks, while the rats in control and MI groups were given no Leonurine.

Two days after ligation, surviving rats were performed in four groups of rats in random fashion. Group 1: Sham-operated rats. The rats served as controls and received saline throughout the study. Group 2: Heart failure rats. This group consisted of rats with heart failure that received saline. Group 3: Heart failure plus low dose leonorine (15 mg/kg/day). Group 4: Heart failure plus high dose leonorine (30 mg/kg/day). All treatment was given via intragastric administration. The experimental period was 8 weeks.

To evaluate the effect of Leonurine on HF, catheterizations to arteries and ventricles are performed to record heat rate (HR), mean aortic pressure (MAP), Left Ventricular Systolic Pressure (LVSP), Left Ventricular End-Diastolic Pressure (LVEDP), peak positive first derivative of left ventricular pressure (+dP/dt), peak negative first derivative of left ventricular pressure (−dP/dt). Data from the measurement of the recording were analyzed to observe the effect of Leonurine. Furthermore, plasma cysteine level and plasma ascorbic acid level were measured by capillary electrophoresis.

The results shown in Table 1 indicate that Leonurine can reduce Left Ventricular End-Diastolic Pressure (LVEDP), increase speed of contraction and improve cardiac function. Single factor Chi square analysis: p<0.05. (Table 1).

TABLE 1
Effects of Leonurine on cardiac function in animal heart failure model

| | Sham | HF + Saline | HF + leo | |
|---|---|---|---|---|
| Parameters | (n = 6) | (n = 8) | 15 mg/kg (n = 8) | 30 mg/kg (n = 8) |
| HR, beats/min | 410 ± 22 | 408 ± 78 | 441 ± 29 | 427 ± 78 |
| MAP, mmHg | 106.55 ± 19.36 | 69.05 ± 16.98^{#} | 107.86 ± 31.23* | 92.59 ± 10.91 |
| LVSP, mmHg | 186.73 ± 36.71 | 113.15 ± 12.9^{#} | 143.77 ± 26.82 | 126.37 ± 2.28 |
| LVEDP, mmHg | 3.88 ± 1.54 | 15.54 ± 1.36^{#} | 11.53 ± 1.83* | 13 ± 5.96 |
| dP/dt, mmHg/s | 6139.47 ± 635.55 | 3047.26 ± 322.71^{#} | 4205 ± 412.71* | 4095 ± 433.45* |
| −dP/dt, mmHg/s | −5571.52 ± 626.56 | −2599.37 ± 587.44^{#} | −4368.83 ± 662.12* | −3276.62 ± 283.77 |

Plasma measurements of Leonurine level, cysteine level and ascorbic acid (Vitamin C) level shown in Table 2 indicate that Leonurine can reduce cysteine level and increase ascorbic acid level. Single factor Chi square analysis: (p<0.05). (Table 2).

TABLE 2
Effects of Leonurine on plasma cysteine level and ascorbic acid (Vitamin C)
level in animal heart failure model

| | Sham | HF + Saline | Leonurine | |
|---|---|---|---|---|
| Compound | (n = 4) | (n = 4) | 15 mg/kg (n = 4) | 30 mg/kg (n = 4) |
| Leonurine, μmol/l | 0 | 0 | 7.42 ± 3.13* | 48.04 ± 10.95** |
| Cysteine, μmol/l | 29.8 ± 2.29 | 55 ± 14.31^{#} | 31.3 ± 21.32* | 35.29 ± 9.73 |
| Ascorbic acid, μmol/l | 11.63 ± 1.66 | 8.42 ± 2.93 | 26.02 ± 3.95** | 39.79 ± 4.33** |

The foregoing examples illustrate certain exemplary embodiments from which other embodiments, alternatives, variations, and modifications will be apparent to those skilled in the art. Accordingly, the invention is intended to embrace all other such alternatives, modifications, and variations that fall within the spirit and scope of the appended claims.

What is claimed is:

1. A method of treating a disease characterized as ischemic cardiomyopathy, comprising administering to a subject suffering from said disease a pharmaceutical composition comprising therapeutically effective amount of synthesized Leonurine of Formula I:

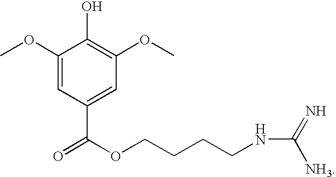

2. The method of claim 1, wherein the subject is a human.

3. The method of claim 1, wherein the subject is hypoxia myocardial cells.

4. The method of claim 1, wherein the pharmaceutical composition further comprises at least one pharmaceutically acceptable carrier.

5. The method of claim 1, wherein the composition is in a form selected from the group consisting of oral form, intravenous form, subcutaneous form, inhalation, and intramuscular form.

6. The method of claim 1, wherein Leonurine induces at least one biochemical changes to improve hypoxia myocardial cells survival rate, reduce LDH releasing from the ischemic myocardial cells, and reduce infarction area of cardiomyopathy in the subject.

7. The method of claim 6, wherein said biochemical change is selected from the group consisting of increasing SOD activity, increasing Catalase activity, reducing MDA level, reducing CK level, reducing LDH level, reducing Bax expression, increasing Bcl-2 expression, and reducing cell apoptosis in cardiac muscle cells.

8. The method of claim 6, wherein said biochemical change is increasing ascorbic acid level in plasma.

9. The method of claim 6, wherein said biochemical change is reducing cysteine level in plasma.
